(19) United States
(12) Patent Application Publication
(10) Pub. No.: US 2006/0135898 A1
(43) Pub. Date: Jun. 22, 2006

(54) Pelvic brace

(76) Inventor: Martin Richardson, Richmond (AU)

Correspondence Address: MERCHANT & GOULD PC, P.O. BOX 2903, MINNEAPOLIS, MN, 55402-0903, US

(21) Appl. No.: 10/534,786

(22) PCT Filed: Nov. 14, 2003
(86) PCT No.: PCT/AU03/01501

§ 371 Date: Nov. 10, 2005

(30) Foreign Application Priority Data
Nov. 15, 2002 (AU) 2002952684

(51) Int. Cl. A61F 5/00

(52) U.S. Cl. 602/19; 602/5

(57) ABSTRACT

A device for bracing the pelvic region of a patient including plurality of straps for wrapping around the pelvic region of a patient is disclosed. The straps are arranged in a side-by-side relationship and interconnected to each adjacent strap at one or more points to restrict relative movement of the straps at each point of interconnection. When positioned on the patient, one of the straps may be an upper strap covering the upper pelvic region and adjoining lower abdominal region of a patient and the other strap or one of the other straps may be a lower strap covering the lower pelvic region and adjoining crutch region of the patient. Fasteners for holding the straps wrapped around the pelvic region of the patient are also included. In use, the fasteners of the upper strap can be released to provide access to the lower abdominal region, or the fasteners of the lower strap can be released to provide access to the crutch region of the patient.

### FIELD OF THE INVENTION

The present invention relates to a device for bracing the pelvic region of a patient.

### BACKGROUND OF THE INVENTION

Casualties of high impact accidents sometimes sustain a fractured or broken pelvis or pelvic bones. One of the many consequences that can occur from a broken pelvic bone is internal bleeding from ruptured blood veins and arteries passing through the pelvic region, such as those conveying blood to or from a patient's legs. The combination of a broken pelvic bone and ruptured blood vessels can result in up to 3 to 6 litres of blood loss into the pelvic region. Blood loss of this magnitude is a serious life threatening condition.

Although advanced surgical procedures have been developed to repair ruptured blood vessels, it is not practical for these surgical procedures to be carried out at an accident scene or on route to a hospital. Paramedics and emergency hospital staff are therefore trained to identify internal bleeding caused by a fractured or broken pelvic bone and to apply temporary measures until surgery can be performed. According to current theory, the bleeding can be minimized by reducing the volume and closing fractured bone ends to tamponade fracture haematocele. In practice, attempts to reduce internal bleeding have included tying bed sheets round the pelvis of a patient. Problems with this technique include firstly, it can be time consuming to apply and secondly, it may require the sheet to be maneuvered beneath a patient, which is usually not possible if the patient is suspected to have also sustained head, neck or spinal injuries.

U.S. Pat. No. 6,240,923 describes a pelvis immobilizing device for applying positive pressure to the pelvis of a patient. The device comprises: a) a wide band of material for wrapping around the pelvic region of a patient; and b) three external straps attached to the band for securing the band around the pelvic region of the patient. Each strap is longer than the band and is provided with an adjustable buckle to tightened the straps and thus apply positive pressure to the pelvic region of the patient. The band is inelastic in a direction along its length and is elastic in a direction along its width to allow the band to conform to the contours of the patient's pelvis. As can be seen in FIG. 3 of the US patent, the width of the band covers the entire pelvic region, including adjoining abdominal and perineum crutch regions of the patient to ensure that the device applies adequate pressure to the pelvis of the patient. Therefore, although the configuration of the device enables it to effectively apply positive pressure to the pelvic region, it may inhibit the treatment of other injuries.

It is therefore an object of the present invention to provide an improved device for bracing the pelvic region of a patient.

### SUMMARY OF THE INVENTION

According to the present invention there is provided a device for bracing the pelvic region of a patient, the device including;

a) a plurality of straps for wrapping around the pelvic region of a patient, the straps being arranged in a side-by-side relationship and interconnected to each adjacent strap at one or more points to restrict relative movement of the straps at the or each point of interconnection, wherein when positioned on the patient, one of the straps may be an upper strap covering the upper pelvic region and adjoining lower abdominal region of a patient and the other strap or one of the other straps may be a lower strap covering the lower pelvic region and adjoining crutch region of the patient; and

b) fastening means on the straps for holding the straps wrapped around the pelvic region of the patient.

In use, the fastening means of the upper strap can be released to provide access to the lower abdominal region, or the fastening means of the lower strap can be released to provide access to the crutch region of the patient.

The present invention therefore provides the advantage that the upper strap can be removed to allow a surgeon to perform surgery to the lower abdominal region while a lower strap or straps remain in position and applies pressure to the pelvic region of the patient. Similarly, if access to the upper thigh or crutch region is required, for example to enable a urinal catheter to be inserted, the lower strap can be removed while the upper strap or straps remain in position and applies pressure to the pelvic region of the patient.

It is preferred that the device include 4 to 8 straps of which 2 to 4 straps may be required to brace the patient's pelvis. The additional straps enable the device to be laid on a hospital bed or trolley such that a patient can be placed on the bed without requiring the device and patient to be maneuvered into alignment. For example, if the patient is placed toward the foot end of the bed the first 2 or more straps of the device may be wrapped around the patient. Similarly, if the patient is placed in the middle of the bed or toward the bed head, the central straps or last 2 or more straps may be used respectively.

Although 2 straps may cover a pelvic region of a child, 3 or 4 straps may be required to cover the pelvic region of an adult. In the instance when 3 or 4 straps are required, it is preferable, in use, that central straps, interposed between the upper and lower straps, remain in position while either the upper or lower strap be released. If necessary, the or each central strap may also be released temporarily to facilitate surgical procedures.

It is preferred that each strap be at least 4 cm wide. It is even more preferred that each strap be at least 6 cm wide. It is even still further preferred that each strap be between 9 and 12 cm wide.

It is preferred that-the straps be interconnected by a resiliently flexible member and that the straps extend from the member in a parallel relationship. It is even more preferred that the resiliently flexible member interconnect the straps at a point along their length that, in use, can be wrapped around the pelvic region of a patient. This preferred aspect of the invention applies irrespective of whether the entire length of the strap or only a portion of each strap is used to wrap around the pelvic regions of the patient.

It is preferred that the fastening means includes a first attachment means and a plurality of co-operating second attachment means spaced along the length of the strap which can be coupled together to form loops of varying sizes.

It is preferred that the first attachment means be in the from of a flexible tab and that the second attachment means be in the form of openings through which the tab can be threaded and fastened.

Although it is possible that the tab may be constituted by a portion of each strap and tied to an opening, it is preferred that the each tab include sections of hook or loop fasteners and that the openings be rings projecting from the straps, whereby in use, the tab can be threaded through a ring and folded on itself so that the hook and loop fasteners engage.

It is preferred that the device further include releasable interconnecting means that interconnect adjacent straps at a spacing from the resiliently flexible member. It is even more preferred that the releasable interconnecting means be in the form of flaps of co-operating hook and loop fasteners that extend from adjacent straps and that the flaps overlap in fastening engagement. The releasable interconnecting means assist in holding the straps in substantially parallel arrangement and may prevent the straps from overlapping.

It is preferred that at least two sets of releasable interconnecting means be spaced along the straps.

In use, it is preferred that the releasable interconnecting means may interconnect the straps wrapped around a patient. It is also preferred that, in use, the releasable interconnecting means of the straps wrapped around the patient be release from the releasable interconnecting means of the adjacent unused straps.

It is preferred that the straps be substantially equal in length and that the resiliently flexible member be located toward an end of the straps and that the each strap be interconnected by the releasable interconnecting means to adjacent straps at 2 or more positions along the length of the straps.

It is preferred that the fastening means of each strap enable the length of the strap wrapped around the patient to be adjusted, thereby allowing the brace to be fitted to patients of varying sizes and allowing the pressure applied by the strap to the patient to be adjusted.

### BRIEF DESCRIPTION OF THE DRAWINGS

A preferred embodiment of the present invention will be described in detail with reference to the accompanying drawings, of which:

FIG. 1 illustrates a front view of a brace having a plurality of straps in a flat orientation;

FIG. 2 illustrates a back view of the device shown in FIG. 1;

FIG. 3 illustrates a front perspective view of the device having three straps in a curled orientation as if wrapped around a patient (not illustrated);

FIG. 4 illustrates an enlarged view of one of the curled straps shown in FIG. 3;

FIG. 5 illustrates a view of the strap shown in FIG. 3 in an alternative arrangement; and

FIG. 6 illustrates a partially exploded cross-sectional view along line a-a in FIG. 2.

### DETAILED DESCRIPTION OF A PREFERRED EMBODIMENT OF PRESENT INVENTION

The brace according to the preferred embodiment includes six straps generally identified by reference numeral 10 arranged in a side-by-side relationship. Each strap 10 is substantially equal in length and has a fastening means for securing each strap in a wrapped orientation around the pelvic region of a patient. Each strap 10 is made from a highly elastic material and is approximately 10cm wide in an unstretched condition.

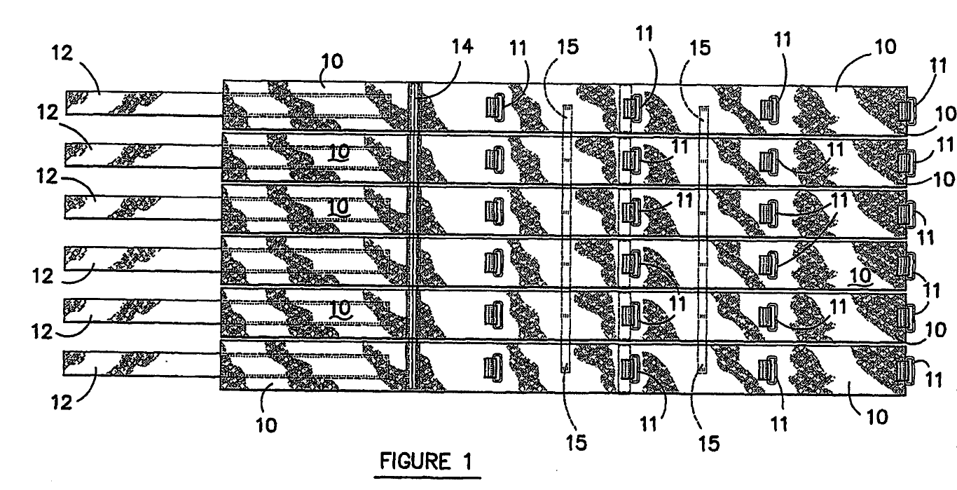

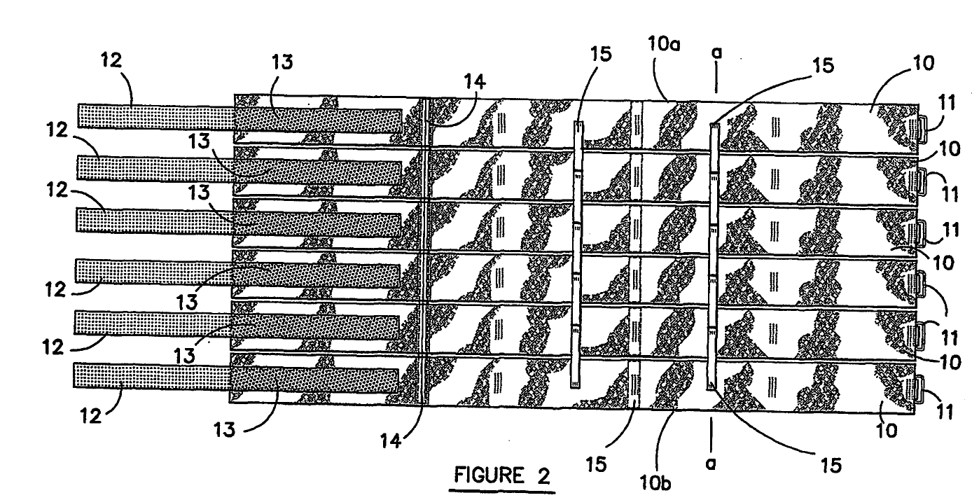

As can best be seen in FIGS. 1, 4 and 5, the fastening means includes a series of D-shaped rings 11 projecting from the front face of each strap 10, and hook and loop fasteners at one end of each strap 10. The hook and loop fasteners are constituted by two separate sections, wherein one section is in the form of a flexible tab 12 that projects from the straps 10 and the other section is a panel 13 sewn in a fixed position over an adjacent portion of the strap 10. Both the tabs 12 and fixed panels 13 have either hook or loop formations that face downwardly or upwardly as shown in FIGS. 1 and 2 respectively.

The brace further includes a resiliently flexible member 14, such as a fiber glass batten or other suitable material that spans across and interconnects the straps 10. The resiliently flexible member 14 is contained within a closed fabric sleeve that is sewn to the straps 10 so that the straps extend in a substantially parallel relationship from the resilient flexible member 14.

In addition, the back face of the device, seen in FIG. 2, includes a series of releasable interconnecting flaps 15 that are sewn to and project laterally of each strap 10. As can be seen in FIG. 6, the top and bottom straps 10a and 10b respectively have one flap 15 that projects inwardly. The straps 10c, 10d, 10e and 10f interposed between the top and bottom straps 10a and 10b include pairs of flaps 15, each flap 15 projecting in opposite directions so as to overlap with a flap 15 of an adjacent strap 10. Each flap 15 includes co-operating hook and loop fasteners for fixing the straps 10 relative to each other.

As can be seen in FIG. 2, two rows of hook and loop flaps 15 are provided in space relationship on the back face of the straps 10.

The width of each strap 10 is such that 2 or more straps 10 can be wrapped around the pelvic region of most patient's involved in high impact accidents. More specifically, the width of 2 adjacent straps 10 is sufficient to cover the pelvic region of a child patient, whereas three or more straps 10 may be required to cover the pelvic region of an adult patient.

In use, the brace can be laid out on a hospital bed or trolley in a flat orientation with the front face facing upwardly as shown in FIG. 1. On account of the surplus number of straps 10, the patient can then be placed on the bed without aligning the patient over the brace. For instance, if the patient is placed toward the foot end of the bed, the first 2 or more straps 10 of the device may be used around the patient. However, if the patient is placed in the middle of the bed the central straps 10 may be used to wrap around the pelvic region of a patient.

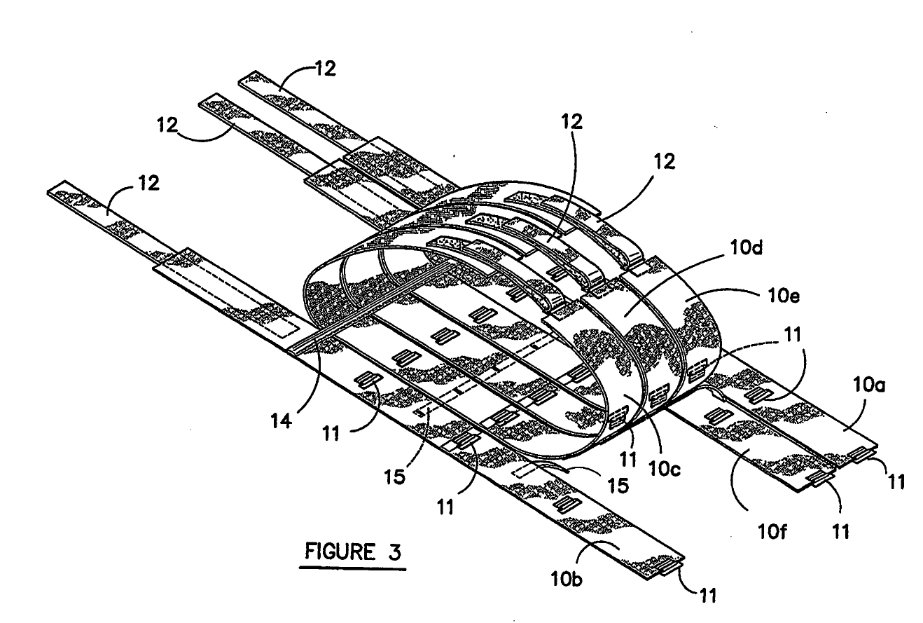

FIG. 3 illustrates three central straps 10c, 10d and 10e in a curled orientation as if wrapped around the pelvic region of a patient. The straps 10 are intended to be held in a wrapped position around the patient by the fastening means, not illustrated in FIG. 3.

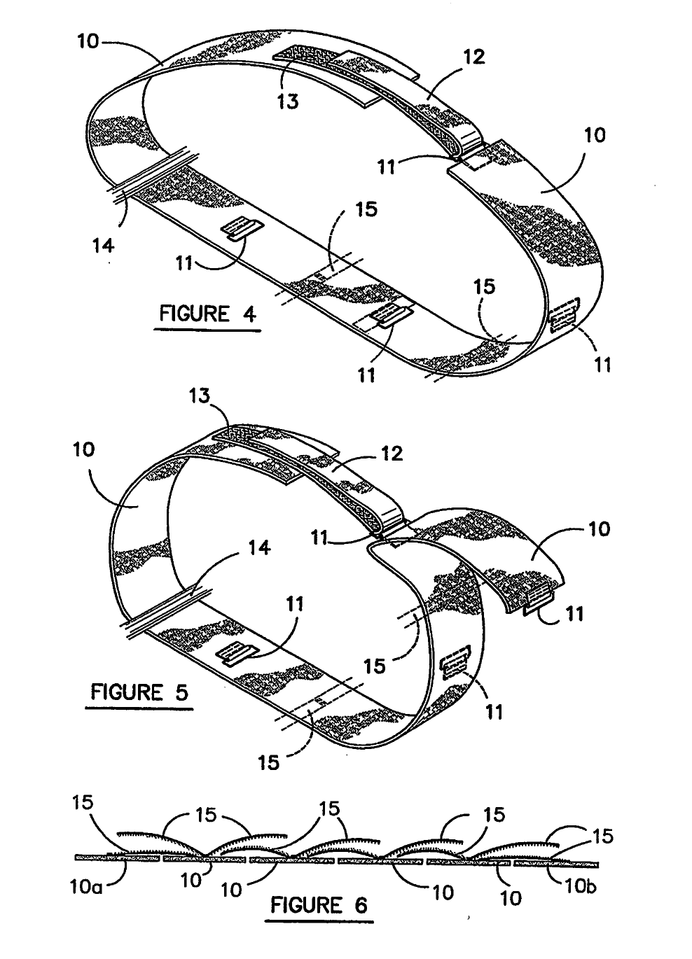

FIGS. 4 and 5 provide an enlarged view of a single strap 10 as if wrapped around a patient. Specifically, FIG. 4 illustrates a loop for wrapping around the pelvic region of a large adult patient, in which the tab 12 extending from one end of the strap is threaded through the D-shaped ring 11 projecting from the opposite end of the strap 10 and folded on itself so that co-operating hook and loop sections are fastened together.

FIG. 5 illustrates a view of a single strap 10 similar to that illustrated in FIG. 4 however, the strap 10 is used to form a loop that can be used to wrap around the pelvic region of a smaller patient. In this instance, the tab 12 is used in the same manner as in FIG. 4 but is threaded through one of the 3 D-shaped rings 11 located inwardly of the end of the strap 10.

The releasable flaps 15, not illustrated in FIG. 3, are intended to prevent the straps from spreading apart. In addition, to facilitate easier operation of the brace, the flaps 15 of the straps 10c, 10d and 10e wrapped around the pelvic region of a patient can be released from adjacent unused straps 10a and 10b. For example, flaps interconnecting the fourth and fifth straps 10e and 10f and interconnecting the first and second straps 10b and 10c of the brace illustrated in FIG. 3 could be released to allow the unused straps 10a, 10b and 10f to remain in a flat orientation.

It will be understood by those skilled in the art of the present invention that modifications may be made without departing from the spirit and scope of the present invention.

1. A device for bracing the pelvic region of a patient, the device including:
a) a plurality of straps for wrapping around the pelvic region of a patient, the straps being arranged in a side-by-side relationship and interconnected to each adjacent strap at one or more points to restrict relative movement of the straps at the or each point of interconnection, wherein when positioned on the patient, one of the straps may be an upper strap covering the upper pelvic region and adjoining lower abdominal region of a patient and the other strap or one of the other straps may be a lower strap covering the lower pelvic region and adjoining crutch region of the patient; and
b) fastening means for holding the straps wrapped around the pelvic region of the patient, whereby in use, the fastening means of the upper strap can be released to provide access to the lower abdominal region, or the fastening means of the lower strap can be released to provide access to the crutch region of the patient.

2. The device according to claim 1, wherein the device includes 4 to 8 straps of which 2 to 4 straps may be required to brace the patient's pelvis.

3. The device according to claim 2, wherein when 3 or 4 straps are required to brace the patient's pelvis, straps interposed between the upper and lower straps can remain in position on the patient while the upper and/or lower strap is released to facilitate surgical procedures.

4. The device according to claim 1, wherein each strap is at least 4 cm wide.

5. The device according to claim 1, wherein each strap is between 9 and 12 cm wide.

6. The device according to claim 1, wherein the straps are interconnected by a resiliently flexible member and that the straps extend from the member in parallel relationship.

7. The device according to claim 6, wherein the resiliently flexible member interconnect the straps at a point along their length that, in use, can be wrapped around the pelvic region of a patient.

8. The device according to claim 5, wherein the straps are further interconnected by releasable interconnecting means that interconnect adjacent straps at a spacing from the resiliently flexible member.

9. The device according to claim 8, wherein the releasable interconnecting means is in the form of flaps of co-operating hook and loop fasteners that extend from adjacent straps and that the flaps overlap in fastening engagement.

10. The device according to claim 8, wherein at least two sets of releasable interconnecting means are spaced along the straps.

11. The device according to claim 8, whereby in use the releasable interconnecting means may interconnect the straps wrapped around the patient.

12. The device according to claim 8, whereby in use the releasable interconnecting means of the straps wrapped around the patient are released from the releasable interconnecting means of adjacent unused straps.

13. The device according to claim 8, wherein the straps are substantially equal in length and that the resiliently flexible member is located toward an end of the straps and that the each strap is interconnected by the releasable interconnecting means to adjacent straps at 2 or more positions along the length of the straps.

14. The device according to claim 1, wherein the fastening means enables the length of each strap wrapped around the patient to be adjusted, thereby allowing the device to be fitted to patients of varying sizes and allowing pressure applied by each strap to the patient to be adjusted.

15. The device according to claim 1, wherein on each strap the fastening means includes a first attachment means and a plurality of co-operating second attachment means spaced along the length of the strap which can be coupled together so that each strap can form a loop of varying sizes.

16. The device according to claim 14, wherein the first attachment means is in the from of a flexible tab and that the second attachment means is in the form of one or more openings through which the tab can be threaded and fastened.

17. The device according to claim 15, wherein each tab includes sections of hook or loop fasteners and that the openings are rings projecting from the straps, whereby in use, the tab can be threaded through a ring and folded on itself so that the hook and loop fasteners engage.
